(19) United States
(12) Patent Application Publication
(10) Pub. No.: US 2024/0325763 A1
(43) Pub. Date: Oct. 3, 2024

(54) AUTOMATIC BACK-UP PACING FROM THE ANODE OF A DEEP SEPTAL LEAD

(71) Applicant: Cardiac Pacemakers, Inc., St. Paul, MN (US)

(72) Inventors: Jessica Burr, Boston, MA (US); Keith L. Herrmann, Minneapolis, MN (US); David L. Perschbacher, Blaine, MN (US)

(21) Appl. No.: 18/620,100

(22) Filed: Mar. 28, 2024

Related U.S. Application Data
(60) Provisional application No. 63/455,390, filed on Mar. 29, 2023.

(51) Int. Cl. A61N 1/37; A61N 1/368

(52) U.S. Cl. CPC A61N 1/3712; A61N 1/3688

(57) ABSTRACT

A method of controlling operation of a medical device includes delivering electrical cardiac pacing energy to a left conduction bundle branch (LBB) area of the subject according to a primary pacing mode that uses a tip electrode of an implantable cardiac lead connected to the medical device for cathodal capture in the LBB area; monitoring for loss of cathodal capture of the LBB area when delivering the electrical cardiac pacing energy in the primary pacing mode; and changing to a backup pacing mode when the loss of cathodal capture is detected, wherein the backup pacing mode uses a ring electrode of the implantable cardiac lead for anodal capture in an interventricular septum of the subject.

### CLAIM OF PRIORITY

This application claims the benefit of U.S. Provisional Application No. 63/455,390 filed on Mar. 29, 2023, which is hereby incorporated by reference in its entirety.

### TECHNICAL FIELD

This document relates generally to cardiac rhythm management systems and particularly, but not by way of limitation, to methods, systems, and devices for providing electrical pacing therapy to the conduction system of the heart.

### BACKGROUND

The heart is the center of a person's circulatory system and includes an intrinsic electro-mechanical system for performing two major pumping functions. The left portions of the heart, including a left atrium (LA) and a left ventricle (LV), draw oxygenated blood from the lungs and pump it to body organs to provide the organs with their metabolic need for oxygen. The right portions of the heart, including a right atrium (RA) and a right ventricle (RV), draw deoxygenated blood from the body organs and pump it to lungs where the blood gets oxygenated. These pumping functions result from contractions of the myocardium of the heart. In a normal heart, a sinoatrial (SA) node, the heart's natural pacemaker, generates intrinsic electrical pulses that propagate through an electrical conduction system to various regions of the heart to excite the myocardial tissues of the cardiac muscles. For example, intrinsic electrical pulses originating from the SA node propagate through an atrio-ventricular (AV) node that is between the RA and RV. From the AV node, a specialized intrinsic conduction system is used by the electrical impulses to reach ventricular myocardial tissues, resulting in contraction activities of ventricles. This specialized conduction system includes the His bundle, the right and left conduction bundle branches that extend along the septum between the RV and LV, and the purkinje fibers that contact the ventricular myocardial tissues.

Coordinated delays of the propagations of the intrinsic electrical pulses in a normal electrical conduction system cause the various portions of the heart to contract in synchrony which results in efficient pumping functions. Heart disease can alter the normal intrinsic conduction paths. A blocked or otherwise abnormal electrical conduction can cause the heart to contract dyssynchronously, resulting in poor hemodynamic performance that may diminish the amount of blood supplied to the heart and the rest of the body. For example, a block in conduction of the electrical pulses in either of the left bundle branch or the right bundle branch can cause dyssynchrony among the ventricles (RV and LV) of the heart. Blockage of the normal conduction paths can cause intrinsic electrical pulses to conduct along alternate pathways, which can cause one ventricle to contract later with respect to the other ventricle. In such events of cardiac malfunctioning, cardiac pacing therapy can be provided to resynchronize contractions of the ventricles of the heart.

### SUMMARY

Methods, systems, and devices to provide electrical cardiac pacing therapy to a subject are disclosed. Example 1 includes subject matter (such as a method of controlling operation of an ambulatory medical device) comprising delivering electrical cardiac pacing energy to a left conduction bundle branch (LBB) area of the subject according to a primary pacing mode that uses a tip electrode of an implantable cardiac lead connected to the medical device for cathodal capture in the LBB area; monitoring for loss of cathodal capture of the LBB area when delivering the electrical cardiac pacing energy in the primary pacing mode; and changing to a backup pacing mode when the loss of cathodal capture is detected, wherein the backup pacing mode uses a ring electrode of the implantable cardiac lead for anodal capture in an interventricular septum of the subject.

In Example 2, the subject matter of Example 1 optionally includes the primary pacing mode being a unipolar pacing mode that uses the tip electrode of the implantable cardiac lead and a device electrode included on the medical device, and the backup pacing mode is a unipolar pacing mode that uses the ring electrode of the implantable cardiac lead and the device electrode.

In Example 3, the subject matter of Example 1 optionally includes the primary pacing mode being a bipolar pacing mode between the tip electrode and the ring electrode, and the backup pacing mode is a unipolar pacing mode that uses the ring electrode of the implantable cardiac lead and a device electrode included on the medical device.

In Example 4, the subject matter of Example 1 optionally includes the primary pacing mode being a unipolar pacing mode that uses the tip electrode of the implantable cardiac lead and a device electrode included on the medical device, and the backup pacing mode is a bipolar pacing mode between the ring electrode of the implantable cardiac lead and the tip electrode of the implantable cardiac lead.

In Example 5, the subject matter of one or any combination of Examples 1-4 optionally includes performing, by the medical device, an automatic capture threshold test for anodal capture using the ring electrode, and enabling the change to the backup pacing mode when a capture threshold is found by the automatic capture threshold test for the anodal capture.

In Example 6, the subject matter of one or any combination of Examples 1-5 optionally includes running an automatic capture threshold test with the medical device in the primary pacing mode when detecting the loss of capture of the LBB area, and performing, by the medical device, an automatic capture threshold test when changing to the backup unipolar pacing mode.

In Example 7, the subject matter of one or any combination of Examples 1-6 optionally includes changing to a backup unipolar pacing mode between the ring electrode of the implantable cardiac lead and a device electrode included on the medical device as the backup pacing mode, and performing, by the medical device, an automatic capture threshold test when changing to the backup unipolar pacing mode.

In Example 8, the subject atter of one or any combination of Examples 1-7 optionally includes triggering an alert regarding operation of the medical device in response to the changing to the backup pacing mode.

Example 9 includes subject matter (such as an implantable medical device) or can optionally be combined with one or any combination of Examples 1-8 to include such subject matter, comprising a therapy circuit configured to provide electrical cardiac pacing energy to a left conduction bundle branch (LBB) area of a subject when operatively connected to an implantable cardiac lead that includes a tip electrode configured for placement inf the left bundle branch; a cardiac signal sensing circuit configured to sense cardiac signals when operatively coupled to implantable electrodes; and a control circuit operatively coupled to the therapy circuit and the cardiac signal sensing circuit. The control circuit is configured to initiate delivery of the electrical cardiac pacing energy to the LBB area using a primary pacing vector that includes the tip electrode of the implantable cardiac lead; monitor for loss of cardiac capture by the primary pacing vector; and change delivery of the electrical cardiac pacing energy to a backup pacing vector when the loss of cardiac capture is detected, wherein the backup pacing vector uses a ring electrode of the implantable cardiac lead for anodal capture in an interventricular septum of the subject.

In Example 10, the subject matter of Example 9 optionally includes a device electrode formed on the implantable medical device, and the primary pacing vector being a unipolar pacing vector that includes the tip electrode and the device electrode, and the backup pacing vector being a unipolar pacing vector that includes the ring electrode and the device electrode.

In Example 11, the subject matter of Example 9 optionally includes a device electrode formed on the implantable medical device, and the primary pacing vector being a bipolar pacing vector that includes the tip electrode and the ring electrode, and the backup pacing vector being a unipolar pacing vector that includes the ring electrode and the device electrode.

In Example 12, the subject matter of Example 9 optionally includes a device electrode formed on the implantable medical device, and the primary pacing vector being a unipolar pacing vector that includes the tip electrode and the device electrode, and the backup pacing vector being a bipolar pacing vector that includes the ring electrode and the tip electrode.

In Example 13, the subject matter of one or any combination of Examples 9-12 optionally includes a control circuit configured to perform an automatic capture threshold test for the backup pacing vector, and enable use of the backup pacing vector when a capture threshold for the backup pacing vector is found by the automatic capture threshold test.

In Example 14, the subject matter of one or any combination of Examples 9-13 optionally includes a control circuit configured to perform an automatic capture threshold test for the primary pacing vector when detecting the loss of capture of the LBB area, and change to delivery of the electrical cardiac pacing energy using the backup pacing vector when the automatic capture threshold test fails to find a capture threshold for the primary pacing vector.

In Example 15, the subject matter of one or any combination of Examples 9-14 optionally includes a device electrode formed on the implantable medical device, and the backup pacing vector being a backup unipolar pacing vector that includes the ring electrode and the device electrode. The control circuit is configured to perform an automatic capture threshold test for the backup unipolar pacing vector when changing to the backup unipolar pacing vector.

In Example 16, the subject matter of one or any combination of Examples 9-15 optionally includes a control circuit configured to trigger an alert regarding operation of the medical device in response to the changing to the backup pacing mode.

Example 17 includes subject matter (such as a cardiac rhythm management system) or can optionally be combined with one or any combination of Examples 1-16 to include such subject matter, comprising an implantable lead including a left bundle branch (LBB) pacing electrode configured for placement in an LBB of a subject, and a ring electrode proximal to the LBB pacing electrode; and a medical device for coupling to the implantable lead. The medical device includes a housing containing electronic circuits of the medical device within the housing and a device electrode on the outside of the housing, a therapy circuit configured to provide electrical cardiac pacing energy to the LBB pacing electrode, a sensing circuit configured to sense cardiac signals, and a control circuit operatively coupled to the therapy circuit and the sensing circuit. The control circuit is configured to initiate delivery of the electrical cardiac pacing energy to the LBB area using a primary pacing vector that includes the LBB pacing electrode, monitor for loss of cardiac capture by the primary pacing vector, and change delivery of the electrical cardiac pacing energy to a backup unipolar pacing vector when the loss of cardiac capture by the primary pacing vector is detected, wherein the backup unipolar pacing vector includes the ring electrode and the device electrode.

In Example 18, the subject matter of Example 17 optionally includes the primary pacing vector including the LBB pacing electrode and the device electrode.

In Example 19, the subject matter of one or both of Examples 17 and 18 optionally includes the primary pacing vector including the LBB pacing electrode and the ring electrode.

In Example 20, the subject matter of one or nay combination of Examples 17-19 optionally includes a control circuit configured to determine that delivering electrical pacing energy to the ring electrode produces capture of the right ventricle (RV) of the subject before including the ring electrode in the backup unipolar pacing vector.

This summary is intended to provide an overview of the subject matter of the present application. It is not intended to provide an exclusive or exhaustive explanation of the invention. The detailed description is included to provide further information about the subject matter of the present patent application.

### BRIEF DESCRIPTION OF THE DRAWINGS

FIG. 1 illustrates portions of an example of a Cardiac Rhythm Management (CRM) system.

FIG. 2 illustrates portions of an example of an implantable medical device (IMD) of a CRM system and portions of the environment in which the IMD operates.

FIG. 3 is a block diagram of an example of an IMD of a CRM system.

FIG. 4 is a flow diagram of an example of a method of operating an IMD.

FIG. 5 illustrates portions of another example of an IMD of a CRM system and portions of the environment in which the IMD operates.

FIGS. 6A-6B show a flow diagram of another example of a method of operating an IMD.

### DETAILED DESCRIPTION

Conventional right ventricular (RV) pacing therapy provides pacing pulses to the RV such as to provide relief to a subject suffering from blockage of normal conduction pathways of the right ventricle. Conduction system pacing (CSP) is the direct pacing of the conduction system of the heart, leading to a more physiological activation of the ventricles alternative to traditional right ventricular pacing. CSP therapy allows pacing at multiple positions within the conduction system (e.g., the His Bundle and the Left Bundle Branch) and multiple types of capture can result from pacing at these positions.

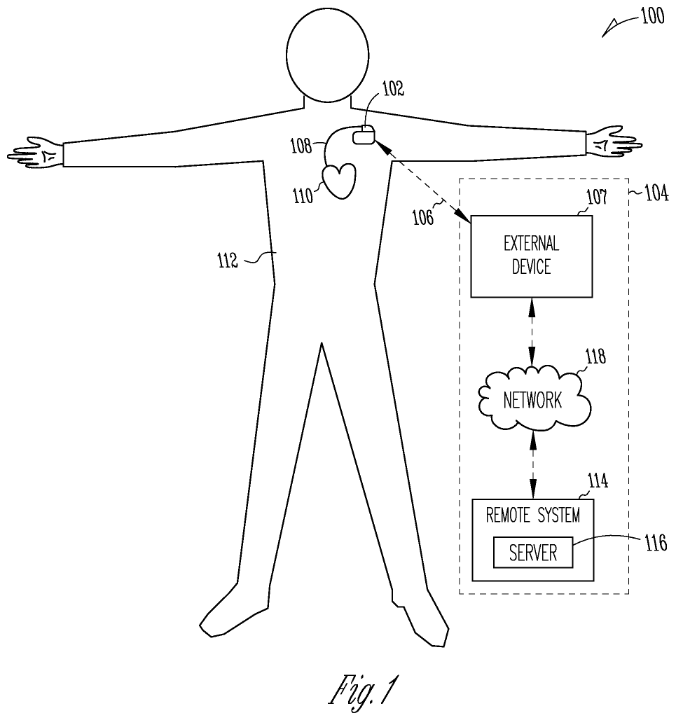

FIG. 1 illustrates portions of an example of a CRM system 100 and portions of an environment in which the CRM system 100 can be used. The CRM system 100 can be configured to include an implantable medical device (IMD) 102 and an external system 104, and a communication link such as a telemetry link 106. The IMD 102 can include an electronic unit coupled by a cardiac lead 108, or additional leads, to a heart 110 of a subject 112. Examples of the IMD 102 can include, but are not limited to, pacemakers, pacemaker/defibrillators, cardiac resynchronization devices, cardiac remodeling control devices, and cardiac monitors. In an example, the IMD 102 can be configured to monitor health of the heart 110 and determine one or more abnormalities associated with the heart 110. The IMD 102 can take a necessary action, such as stimulating one or more portions of the heart 110 through the lead 108, to treat the one or more abnormalities.

In an example, the external system 104 can include an external device 107 configured to communicate bi-directionally with the IMD 102 such as through the telemetry link 106. For example, the external device 107 can include a programmer to program the IMD 102 to provide one or more therapies to the heart 110. In an example, the external device 107 can program the IMD 102 to detect presence of a conduction block in a left bundle branch (LBB) of the heart 110 and prevent dyssynchronous contraction of the heart 110 by providing a cardiac resynchronization therapy (CRT) to the heart 110.

In an example, the external device 107 can be configured to transmit data to the IMD 102 through the telemetry link 106. Examples of such transmitted data can include programming instructions for the IMD 102 to acquire physiological data, perform at least one self-diagnostic test (such as for a device operational status), or deliver at least one therapy or any other data. In an example, the IMD 102 can be configured to transmit data to the external device 107 through the telemetry link 106. This transmitted data can include real-time physiological data acquired by the IMD 102 or stored in the IMD 102, therapy history data, an operational status of the IMD 102 (e.g., battery status or lead impedance), and the like. The telemetry link 106 can include an inductive telemetry link or a far-field radio-frequency telemetry link.

In an example, the external device 107 can be a part of a patient management system that can include other devices such as a remote system 114 for remotely programming the IMD 102. In an example, the remote system 114 can be configured to include a server 116 that can communicate with the external device 107 through a telecommunication network 118 such as to access the IMD 102 to remotely monitor the health of the heart 110 or adjust parameters associated with the one or more therapies.

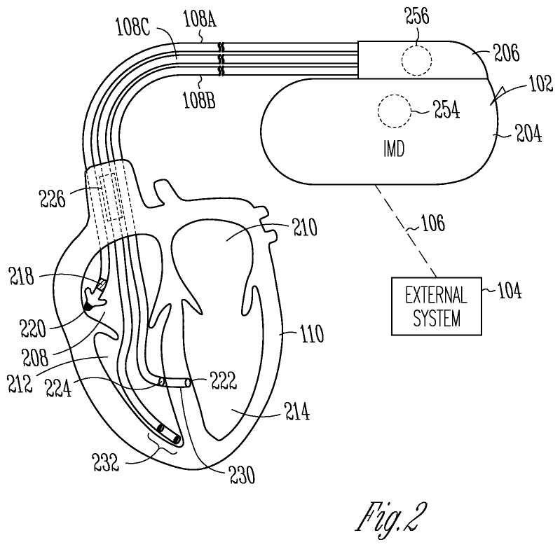

FIG. 2 illustrates portions of another example of an IMD 102 of a CRM system and portions of the environment in which the IMD 102 operates. The IMD 102 can include a hermetically sealed housing 204 or “can” and a header 206 that extends from the housing 204. The header 206 can include one or more receptacles such as for receiving proximal ends of one or more cardiac leads such as a lead 108A, a lead 108B and a lead 108C (collectively referred to herein as leads 108). Distal ends of the leads 108 can be coupled to electrodes such as for providing pacing energy, defibrillation energy, or both, in conjunction with the electrodes disposed on or around the heart 110. The electrodes can also be used for sensing electrical activity of the heart 110, including electrical activity related to contractions of the atria or ventricles.

As shown in FIG. 2, the heart 110 includes a right atrium (RA) 208, a left atrium (LA) 210, a right ventricle (RV) 212, a left ventricle (LV) 214, and a coronary sinus 216 extending from the RA 208. In an example, lead 108A can be an intravascular RA lead that can extend from a superior vena cava (SVC) into the RA 208 and can include electrodes such as a ring electrode 218 and a tip electrode 220 such as for sensing signals, or delivering pacing therapy, or both, to the RA 208.

In an example, lead 108C can be an intravascular right ventricle (RV) lead that can extend from the SVC into the RA 208, and then into the RV 212. The lead 108C can be configured to include a defibrillation coil electrode 226 such as to provide high energy shock therapy to the subject. The RV lead 108C can include an electrode pair 232 for sensing signals, delivering pacing therapy, or both. The RV lead 108C can be configured to achieve resynchronization of the RV 212.

In an example, lead 108B can be a Left Bundle Branch (LBB) area lead that can extend into the RA 208, into the RV 212, and then into the LBB area 230. In an example, the LBB area lead 108B can include tip electrode 222 and ring electrode 224 that can be used to deliver electro-stimulation energy and to sense intrinsic cardiac signals of the heart 110.

The IMD 102 can include a device electrode on the exterior of the IMD 102. The device electrode can be a can electrode 254 that includes at least a portion of the outside of the housing 204 or a header electrode 256 included in the header 206 of the IMD 102. The IMD 102 can provide unipolar pacing using a unipolar pacing vector that includes the device electrode and any pacing electrode of the leads. The IMD 102 can provide bipolar pacing using a bipolar pacing vector that includes a bipolar electrode pair (e.g., tip electrode 222 and ring electrode 224). The IMD 102 can use unipolar sensing to sense intrinsic electrical cardiac signals using the device electrode and a lead electrode or use bipolar sensing to sense intrinsic electrical signals using bipolar electrode pairs.

The IMD 102 can provide electrical stimulation pulses to a stimulation location in the LBB area 230. Left Bundle Branch Area Pacing (LBBAP) is the pacing capture of the LBB via the interventricular septum. LBBAP can be an alternative to traditional RV pacing and can be used for cardiac resynchronization therapy (CRT) in which cardiac stimulation is used to provide proper synchronization of the depolarizations of the ventricles. LBBAP involves the placement of LBBAP lead 108B into the LBB on the left side of the interventricular septum. Bipolar LBBAP can be delivered using tip electrode 222 as the pacing cathode and ring electrode 224 as the pacing anode. Unipolar pacing can be delivered tip electrode 222 as the pacing cathode and a device electrode (e.g., can electrode 254) as the pacing anode.

Because LBBAP is relatively new and non-traditional, physicians may prefer to include the RV pacing lead 108C only to provide backup pacing that may be needed when there is an increase in pacing threshold, dislodgement of the LBBAP lead 108B, or perforation of the LBBAP electrode into the LV. Backup pacing capability that does not use the RV lead 108C would remove the need for the additional RV backup lead.

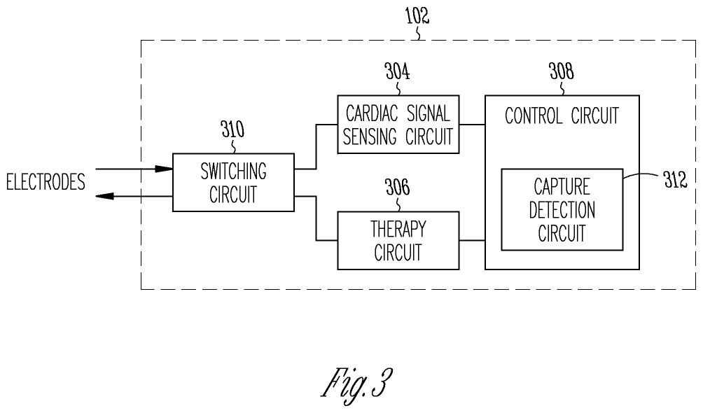

FIG. 3 is a block diagram of portions of an example of an IMD 102 of a CRM system (e.g., CRM system 100 of FIG. 1) that provides backup pacing to the RV without an additional RV lead (e.g., without RV lead 108C in FIG. 2). The IMD 102 can include a cardiac signal sensing circuit 304, a therapy circuit 306, and a control circuit 308. The IMD 102 can be connected to at least one implantable cardiac lead. The implantable cardiac lead can include an LBBAP electrode configured for placement in or near the LBB of a patient (e.g., tip electrode 222 in FIG. 2) and another lead electrode proximal to the LBBAP electrode (e.g., ring electrode 224 in FIG. 2). The IMD 102 includes a device electrode (e.g., can electrode 254 in FIG. 2).

The therapy circuit 306 provides electrical pacing energy to the LBB of the patient when operatively connected to pacing electrodes of the system including the LBBAP electrode. The cardiac signal sensing circuit 304 includes one or more sense amplifiers to sense intrinsic cardiac signals and paced cardiac signals when coupled to electrodes of the system including the LBBAP electrode. Switching circuit 310 switches different combination of electrodes to the therapy circuit 306 and cardiac signal sensing circuit 304 to change the electrode configurations used in delivering cardiac pacing energy and sensing cardiac signals.

The control circuit 308 is operatively coupled to the therapy circuit 306 and cardiac signal sensing circuit 304, and may include a digital signal processor, application specific integrated circuit (ASIC), field programmable gate array (FPGA), microprocessor, or other type of processor, interpreting or executing instructions in software or firmware. In some examples, the control circuit 308 may include a state machine or sequencer that is implemented in hardware circuits. The control circuit 308 may include any combination of hardware, firmware, or software. The control circuit 308 includes one or more circuits to perform the functions described herein. A circuit may include software, hardware, firmware, or any combination thereof. For example, the circuit may include instructions in software executing on the control circuit 308. Multiple functions may be performed by one or more circuits.

The control circuit 308 includes a capture detection circuit 312 configured to detect cardiac capture. The capture detection circuit 312, as part of an automatic capture threshold test, delivers electrostimulation energy using a first energy level, and changes the electrostimulation energy level by at least one of: a) increasing the electrostimulation energy from the first energy level until detecting that the electrostimulation energy induces cardiac capture, or b) reducing the electrostimulation energy from the first energy level until detecting that the stimulation energy fails to induce cardiac capture. The capture detection circuit 312 continues the changing of the stimulation energy level until confirming the inducement of stable capture or the failure to induce capture.

The control circuit 308 may then derive an electrostimulation energy value for cardiac pacing using a determined minimum energy that induces stable capture. In some examples, this derived electrostimulation energy value may be the minimum amplitude of the energy that induced stable capture, or the minimum amplitude plus a safety margin. The control circuit 308 may then set the electrostimulation energy for pacing using the derived electrostimulation energy value.

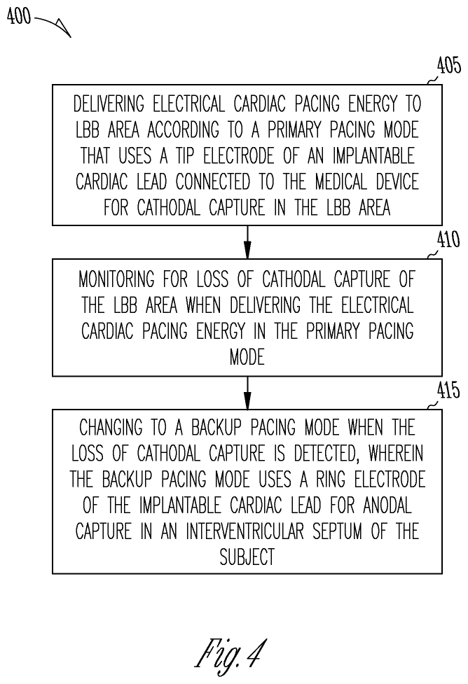

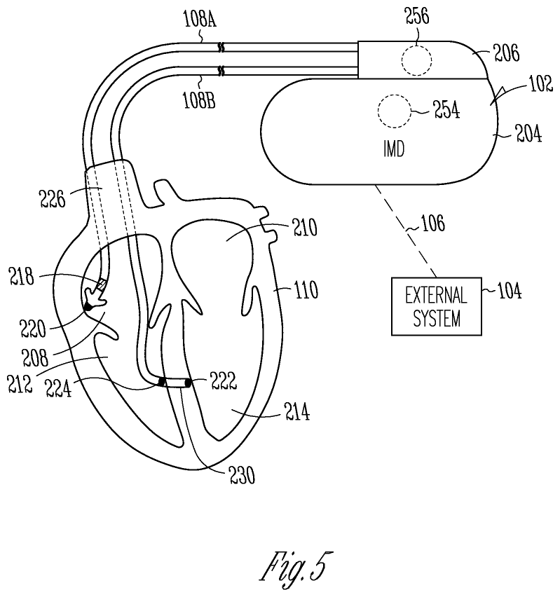

FIG. 4 is a flow diagram of a method 400 of controlling operation of a medical device to provide electrical cardiac pacing therapy to a subject. The method can be performed using the IMD 102 of FIG. 5 that does not include an RV lead or an RV apex electrode. The IMD 102 can deliver electrical cardiac pacing energy according to a primary pacing mode and a backup pacing mode.

At block 405, the medical device delivers electrical cardiac pacing energy to the LBB area of the patient according to a primary pacing mode. The primary pacing mode is an LBBAP mode that uses a primary pacing vector that includes an LBBAP electrode (e.g., lead tip electrode 222 in FIG. 5). The primary LBBAP mode provides pacing with cathodal capture. The primary LBBAP vector may be a unipolar pacing vector that includes the LBBAP electrode as the pacing cathode and a device electrode (e.g., can electrode 254 or header electrode 256 in FIG. 5) as the pacing anode. In some examples, the primary LBBAP vector is a bipolar pacing vector that includes a bipolar electrode pair with the LBBAP electrode (e.g., tip electrode 222 in FIG. 5) as the pacing cathode and a lead electrode proximal to the LBBAP electrode (e.g., ring electrode 224 in FIG. 5) as the pacing anode.

This LBB pacing area 230 can range from two to ten times deeper than the traditional right ventricular pacing target (e.g., the RV apex), depending on the thickness of the interventricular septum. With the increased septal depth of the placement of the LBBAP electrode, the lead electrode proximal to the LBBAP electrode may be in contact the septal tissue or even be embedded in the septal tissue as well. This electrode can be used for backup pacing. For instance, in the example of FIG. 5, the tip electrode 222 is in the LBB area 230 and the ring electrode 224 may be in contact with the septum or may be within the septum. The ring electrode 224 can be repurposed for backup pacing in the event of loss of capture with the tip electrode 222.

The septal backup pacing vector can be a unipolar pacing vector that includes the ring electrode and the device electrode. In some examples, the unipolar pacing vector provides anodal capture with the ring electrode as the pacing anode and the device electrode as the pacing cathode in the backup pacing mode. Other backup pacing vectors are possible. For example, the backup pacing vector can be a septal bipolar pacing vector that includes the ring electrode and the tip electrode. The septal backup bipolar pacing vector may provide anodal capture using the ring electrode as the pacing anode.

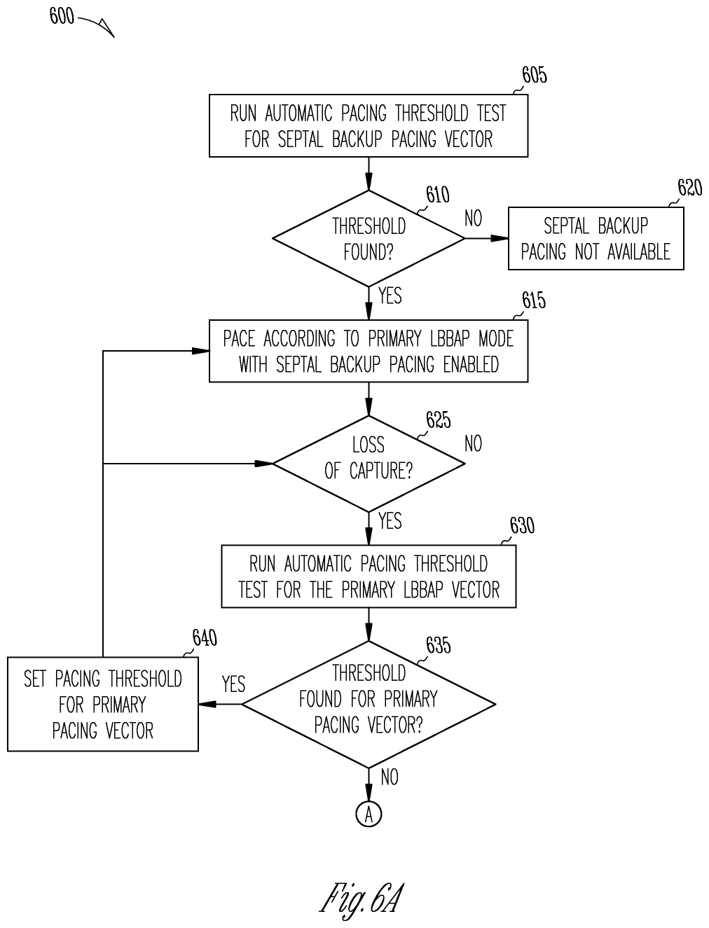

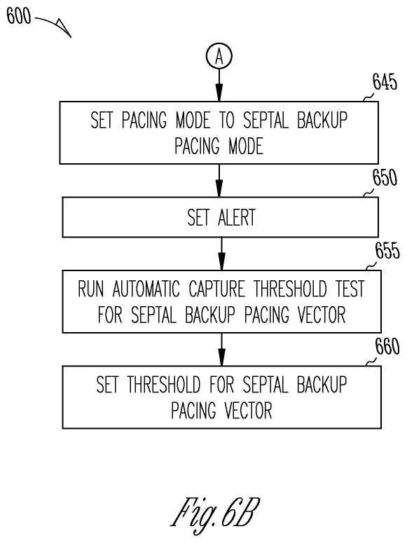

FIGS. 6A and 6B is a flow diagram of an example of a method 600 of controlling operation of a medical device to configure a septal backup pacing mode to a primary LBBAP mode. Before activating the septal backup pacing mode, it may be helpful to verify that the septal backup pacing vector is available for use. For example, if the septal backup pacing vector includes the lead ring electrode (e.g., ring electrode 224 in FIG. 5) and a can electrode (e.g., can electrode 254 in FIG. 5), it may be helpful to verify that anodal capture occurs at the ring electrode.

One approach to verify anodal capture at the ring electrode is for an external device (e.g., external device 107 in FIG. 1) to prompt the clinician to verify that RV capture is present when pacing using the septal backup pacing vector. The clinician may program the IMD 102 to pace using the ring electrode and verify the capture using a surface electrocardiograph (ECG) for example. The clinician affirmatively responds to the prompt if the ECG shows capture by the ring electrode. The IMD 102 then activates or enables use of the septal backup pacing vector. At block 605, the control circuit of the IMD 102 may run an automatic capture threshold test to find an appropriate pacing energy for the septal backup pacing vector.

In some examples, the IMD 102 includes a capture detection circuit (e.g., capture detection circuit 312 in FIG. 3) that can detect anodal capture. Electrostimulation can be either cathodal or anodal, or a combination of anodal and cathodal. In cathodal stimulation, a more negative electrode induces capture of the heart chamber. In anodal stimulation, a more positive electrode induces the capture. Anodal stimulation can be detected by the absence of a delay between a bipolar stimulation pulse and an evoked response sensed at the electrode functioning as the anode during the stimulation. If a capture threshold for the ring electrode is found at block 610, the IMD 102 may store the determined backup pacing energy. At block 615, the IMD 102 delivers LBBAP according to the primary LBBAP mode with septal backup pacing mode enabled.

Another approach to verify anodal capture at the ring electrode is for the IMD 102 to run the automatic capture threshold test for the septal backup pacing vector at block 605 and detect anodal RV capture using the test. If anodal RV capture is not detected, a capture threshold is not found at block 610 and the threshold test will fail. At block 620, it is determined that septal backup pacing is not available. The external device may indicate this fact on the user interface, such as by graying out the feature on the display of the external device. If anodal RV capture is detected and a capture threshold is found, at block 615, the IMD 102 delivers LBBAP according to the primary LBBAP mode with the septal backup pacing mode enabled.

At block 625, with the primary LBBAP mode activated, the IMD 102 recurrently monitors for loss of capture of the LBB area by the primary LBBAP vector. The control circuit of the IMD 102 may set a longer timing window for detection of LBBAP loss of capture (e.g., up to 80 milliseconds) than a timing window for detection of traditional RV pacing loss of capture. This is because of the additional delay in septal capture to myocardial depolarization. The control circuit of the IMD 102 may declare loss of capture when two loss of capture events occur during four pacing deliveries in the LBBAP mode.

At block 630, the control circuit of the IMD 102 runs an automatic capture threshold test for the primary LBBAP vector. This test is performed to see if the primary LBBAP mode can be adjusted to resolve the loss of capture in the LBBAP mode. If a pacing energy capture threshold is found at block 635, the control circuit of the IMD 102 sets the pacing threshold at block 640, and the method 600 returns to block 615 to deliver pacing therapy according to the primary LBBAP mode.

If a pacing energy capture threshold for the LBBAP mode is not found at block 635, the control circuit changes to delivering pacing therapy according to the septal backup pacing mode at block 645. At block 650, the control circuit triggers an alert of the switch to the backup pacing mode. The alert or warning may be displayed the next time the IMS 102 is interrogated by an external device, or the alert may be sent to a personal monitoring device of the patient. The alert may be a warning that the lead may have to be repositioned.

At bock 655, the control circuit of the IMD 102 may run an automatic capture threshold test for the septal backup pacing mode. At block 660, the result of the capture threshold test is used to set an appropriate pacing energy level for the septal backup pacing mode.

Returning to block 635, in some examples, the control circuit of the IMD 102 may be programmed to first try another primary pacing mode before reverting to the anodal septal backup pacing mode. For instance, if the primary LBBAP vector is from tip electrode 222 in FIG. 5 to can electrode 254 in FIG. 5, the IMD 102 may first try pacing using a vector from tip electrode 222 to ring electrode 224 before trying the ring electrode to can electrode backup pacing vector.

The order of attempts may be reversed if the primary LBBAP vector is from tip electrode 222 to ring electrode 224. If the IMD 102 detects loss of capture using a primary pacing vector from tip electrode 222 to ring electrode 224, the IMD 102 may first try pacing using tip electrode 222 to can electrode 254 before reverting to pacing using the backup pacing vector of ring electrode 224 to can electrode 254.

Due to non-traditional nature of CSP, physicians may choose to add an RV lead in the RV apex for backup pacing in the event of dislodgement of the LBB area lead, perforation by an LBBAP electrode into the LV, etc. In lieu of adding an extra lead, the present methods, systems, and devices repurpose the electrodes of the LBBAP lead for backup pacing. This eliminates the need of the extra RV lead.

### Additional Description

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A method of controlling operation of a medical device to provide electrical cardiac pacing therapy to a subject, the method comprising:
delivering electrical cardiac pacing energy to a left conduction bundle branch (LBB) area of the subject according to a primary pacing mode that uses a tip electrode of an implantable cardiac lead connected to the medical device for cathodal capture in the LBB area;
monitoring for loss of cathodal capture of the LBB area when delivering the electrical cardiac pacing energy in the primary pacing mode; and
changing to a backup pacing mode when the loss of cathodal capture is detected, wherein the backup pacing mode uses a ring electrode of the implantable cardiac lead for anodal capture in an interventricular septum of the subject.

2. The method of claim 1, wherein the primary pacing mode is a unipolar pacing mode that uses the tip electrode of the implantable cardiac lead and a device electrode included on the medical device, and the backup pacing mode is a unipolar pacing mode that uses the ring electrode of the implantable cardiac lead and the device electrode.

3. The method of claim 1, wherein the primary pacing mode is a bipolar pacing mode between the tip electrode and the ring electrode, and the backup pacing mode is a unipolar pacing mode that uses the ring electrode of the implantable cardiac lead and a device electrode included on the medical device.

4. The method of claim 1, wherein the primary pacing mode is a unipolar pacing mode that uses the tip electrode of the implantable cardiac lead and a device electrode included on the medical device, and the backup pacing mode is a bipolar pacing mode between the ring electrode of the implantable cardiac lead and the tip electrode of the implantable cardiac lead.

5. The method of claim 1, including:
performing, by the medical device, an automatic capture threshold test for anodal capture using the ring electrode; and
enabling the change to the backup pacing mode when a capture threshold is found by the automatic capture threshold test for the anodal capture.

6. The method of claim 1, wherein the changing to the backup mode includes:
running an automatic capture threshold test with the medical device in the primary pacing mode when detecting the loss of capture of the LBB area; and
changing operation of the medical device to the backup pacing mode when a capture threshold is not found by the automatic capture threshold test.

7. The method of claim 1, wherein changing to the backup pacing mode includes:
changing to a backup unipolar pacing mode between the ring electrode of the implantable cardiac lead and a device electrode included on the medical device as the backup pacing mode; and
performing, by the medical device, an automatic capture threshold test when changing to the backup unipolar pacing mode.

8. The method of claim 1, including triggering an alert regarding operation of the medical device in response to the changing to the backup pacing mode.

9. An implantable medical device, the device comprising:
a therapy circuit configured to provide electrical cardiac pacing energy to a left conduction bundle branch (LBB) area of a subject when operatively connected to an implantable cardiac lead that includes a tip electrode configured for placement inf the left bundle branch;
a cardiac signal sensing circuit configured to sense cardiac signals when operatively coupled to implantable electrodes; and
a control circuit operatively coupled to the therapy circuit and the cardiac signal sensing circuit, and configured to:
initiate delivery of the electrical cardiac pacing energy to the LBB area using a primary pacing vector that includes the tip electrode of the implantable cardiac lead;
monitor for loss of cardiac capture by the primary pacing vector; and
change delivery of the electrical cardiac pacing energy to a backup pacing vector when the loss of cardiac capture is detected, wherein the backup pacing vector uses a ring electrode of the implantable cardiac lead for anodal capture in an interventricular septum of the subject.

10. The device of claim 9, including:
a device electrode formed on the implantable medical device; and
wherein the primary pacing vector is a unipolar pacing vector that includes the tip electrode and the device electrode, and the backup pacing vector is a unipolar pacing vector that includes the ring electrode and the device electrode.

11. The device of claim 9, including:
a device electrode formed on the implantable medical device; and
wherein the primary pacing vector is a bipolar pacing vector that includes the tip electrode and the ring electrode, and the backup pacing vector is a unipolar pacing vector that includes the ring electrode and the device electrode.

12. The device of claim 9, including:
a device electrode formed on the implantable medical device; and
wherein the primary pacing vector is a unipolar pacing vector that includes the tip electrode and the device electrode, and the backup pacing vector is a bipolar pacing vector that includes the ring electrode and the tip electrode.

13. The device of claim 9, wherein the control circuit is configured to:
perform an automatic capture threshold test for the backup pacing vector; and
enable use of the backup pacing vector when a capture threshold for the backup pacing vector is found by the automatic capture threshold test.

14. The device of claim 9, wherein the control circuit is configured to:
perform an automatic capture threshold test for the primary pacing vector when detecting the loss of capture of the LBB area; and
change to delivery of the electrical cardiac pacing energy using the backup pacing vector when the automatic capture threshold test fails to find a capture threshold for the primary pacing vector.

15. The device of claim 9, including:
a device electrode formed on the implantable medical device;
wherein the backup pacing vector is a backup unipolar pacing vector that includes the ring electrode and the device electrode; and
wherein the control circuit is configured to perform an automatic capture threshold test for the backup unipolar pacing vector when changing to the backup unipolar pacing vector.

16. The device of claim 9, wherein the control circuit is configured to trigger an alert regarding operation of the medical device in response to the changing to the backup pacing mode.

17. A cardiac rhythm management system, the system comprising:
an implantable lead including a left bundle branch (LBB) pacing electrode configured for placement in an LBB of a subject, and a ring electrode proximal to the LBB pacing electrode;
a medical device for coupling to the implantable lead, the medical device including:
a housing containing electronic circuits of the medical device within the housing and a device electrode on the outside of the housing;
a therapy circuit configured to provide electrical cardiac pacing energy to the LBB pacing electrode;
a sensing circuit configured to sense cardiac signals; and
a control circuit operatively coupled to the therapy circuit and the sensing circuit, and configured to:
initiate delivery of the electrical cardiac pacing energy to the LBB area using a primary pacing vector that includes the LBB pacing electrode;
monitor for loss of cardiac capture by the primary pacing vector; and
change delivery of the electrical cardiac pacing energy to a backup unipolar pacing vector when the loss of cardiac capture by the primary pacing vector is detected, wherein the backup unipolar pacing vector includes the ring electrode and the device electrode.

18. The system of claim 17, wherein the primary pacing vector includes the LBB pacing electrode and the device electrode.

19. The system of claim 17, wherein the primary pacing vector includes the LBB pacing electrode and the ring electrode.

20. The system of claim 17, wherein the control circuit is configured to determine that delivering electrical pacing energy to the ring electrode produces capture of the right ventricle (RV) of the subject before including the ring electrode in the backup unipolar pacing vector.
